(19) United States
(12) Patent Application Publication
(10) Pub. No.: US 2022/0079821 A1
(43) Pub. Date: Mar. 17, 2022

(54) ARTICLE INCLUDING MICROPOROUS FILM AND METHOD OF MAKING THE SAME

(71) Applicant: 3M INNOVATIVE PROPERTIES COMPANY, St. Paul, MN (US)

(72) Inventors: Neelakandan Chandrasekaran, Plymouth, MN (US); Scott M. Niemi, St. Paul, MN (US); Thomas J. Gilbert, St. Paul, MN (US); Shannon R.A. Harnden, Lake Elmo, MN (US)

(21) Appl. No.: 17/419,538

(22) PCT Filed: Dec. 30, 2019
(86) PCT No.: PCT/US2019/068947

§ 371 Date: Jun. 29, 2021

Related U.S. Application Data
(60) Provisional application No. 62/786,757, filed on Dec. 31, 2018.

(51) Int. Cl. A61F 13/56; A61F 13/15; A61F 13/84; B29C 48/00; B29C 48/08; B29C 48/21; B29C 48/91

(52) U.S. Cl. CPC A61F 13/5622; A61F 13/15756; A61F 13/8405; B29C 48/0018; B29C 48/08; B29K 2101/12; B29C 48/91; A61F 2013/5666; A61F 2013/8402; A61F 2013/8497; B29C 48/21

(57) ABSTRACT

The article includes at least a first layer having first and second major surfaces and a second layer adjacent the second major surface of the first layer. The first major surface of the first layer is at least a portion of a visible, outer surface of the article. At least a portion of the first layer is a see-through thermoplastic film that has a first color other than white. The second layer includes a microporous film having an opaque, microporous region and a nonporous region. When the opaque, microporous region and the nonporous region are seen through the first layer, they create an appearance on the outer surface of two different colors or two different shades of the same color. A method of making the article is also described.

### CROSS-REFERENCE TO RELATED APPLICATION

This application claims priority to U.S. Provisional Application No. 62/786,757, filed Dec. 31, 2018, the disclosure of which is incorporated by reference in its entirety herein.

### BACKGROUND

Film articles having printed and/or colored regions are useful for several different applications. For example, colored duct tape and washi tape have become popular for decorating and craft projects. A variety of different personal hygiene articles (e.g., absorbent articles such as diapers, adult incontinence products, and sanitary napkins) that include different printed and/or colored regions are available in the market. Printing or coloring on these and other articles can be attractive to the consumer and help the consumer differentiate between different brands. Some manufacturers of absorbent articles print with multi-colored graphics that are a signature of their brand. Others may use monochromatic printing on the articles.

Printing approaches to providing a differentiated product generally use ink, colored adhesives, or heat- or pressure-activated chemical colorants, each of which adds cost to the product that is passed on to consumers. Some recent examples of absorbent articles with patterns or colors include those described in U.S. Pat. No. 8,324,444 (Hansson et al.) and U.S. Pat. Appl. Pub. Nos. 2011/0264064 (Arora et al.) and 2012/0242009 (Mullane et al.).

### SUMMARY

The present disclosure can be useful, for example, for providing visual images on products without the need for the printing of inks or other color-providing chemicals. The article includes an opaque, microporous region and a nonporous region. The contrast between opaque, microporous regions and nonporous regions in the article of the present disclosure typically and advantageously provides a durable image that is resistant to fading over time. Furthermore, since the articles include a microporous thermoplastic film, they can block the transmission of light (e.g., by scattering), allowing them to be detected in inspection systems that rely upon shining a light onto a substrate and detecting the amount of light received from the area of the irradiated substrate. Thus, the articles of the present disclosure are useful for facilitating the inspection process of certain manufactured products. The opaque, microporous region and nonporous region have predetermined (in other words, designed) shapes. Advantageously, the regions can be in the form of a wide variety of patterns, numbers, pictures, symbols, alphabetical letters, bar codes, or combinations thereof that can be selected to be decorative or distinguishing. The region can also be in the form of a company name, brand name, or logo that may be readily identified by a customer. The article of the present disclosure can be readily customized depending on the requirements of a particular product.

In one aspect, the present disclose provides an article that includes at least a first layer having first and second major surfaces and a second layer adjacent the second major surface of the first layer. The first major surface of the first layer is at least a portion of a visible, outer surface of the article. At least a portion of the first layer is a see-through thermoplastic film that has a first color other than white. The second layer includes a microporous film having an opaque, microporous region and a nonporous region. When the opaque, microporous region and the nonporous region are seen through the first layer, they create an appearance on the outer surface of two different colors or two different shades of the same color.

In another aspect, the present disclosure provides a method of making the article. The method includes providing a multilayer film including the first layer and a microporous thermoplastic film and collapsing some pores in the microporous film to form the nonporous region.

In this application, terms such as “a”, “an” and “the” are not intended to refer to only a singular entity but include the general class of which a specific example may be used for illustration. The terms “a”, “an”, and “the” are used interchangeably with the term “at least one”. The phrases “at least one of” and “comprises at least one of” followed by a list refers to any one of the items in the list and any combination of two or more items in the list. All numerical ranges are inclusive of their endpoints and non-integral values between the endpoints unless otherwise stated.

The terms “first” and “second” are used in this disclosure in their relative sense only. It will be understood that, unless otherwise noted, those terms are used merely as a matter of convenience in the description of one or more of the embodiments.

The term “microporous” refers to having multiple pores that have an average dimension (in some cases, diameter) of up to 10 micrometers. At least some of the multiple pores should have a dimension on the order of or larger than the wavelength of visible light. For example, at least some of the pores should have a dimension (in some cases, diameter) of at least 400 nanometers. Pore size is measured by measuring bubble point according to ASTM F-316-80. The pores may be open cell pores or closed cell pores. In some embodiments, the pores are closed cell pores.

The term “see-through” refers to either transparent (that is, allowing passage of light and permitting a clear view of objects beyond) or translucent (that is, allowing passage of light and not permitting a clear view of objects beyond).

If the nonporous region is said to be “within” the opaque, microporous region, it means that the opaque, microporous region may border the nonporous region on at least two sides or more. In some embodiments, the opaque, microporous region surrounds the nonporous region. Generally, the nonporous region is not found only at the edge of the microporous film.

The thickness of a film should be understood to be its smallest dimension. It is generally referred to as the “z” dimension and refers to the distance between the major surfaces of the film.

The term “upstanding” with regard to the mechanical fastening elements refers to posts that protrude from the thermoplastic backing and includes posts that stand perpendicular to the backing and posts that are at an angle to the backing other than 90 degrees.

The above summary of the present disclosure is not intended to describe each disclosed embodiment or every implementation of the present disclosure. The description that follows more particularly exemplifies illustrative embodiments. It is to be understood, therefore, that the drawings and following description are for illustration purposes only and should not be read in a manner that would unduly limit the scope of this disclosure.

### BRIEF DESCRIPTION OF THE DRAWINGS

The disclosure may be more completely understood in consideration of the following detailed description of various embodiments of the disclosure in connection with the accompanying drawings, in which:

FIG. 1 is a perspective view of an embodiment of a three-layer construction useful in various embodiments of the article of the present disclosure;

FIG. 2 is a perspective view of an embodiment of a personal hygiene article incorporating an embodiment of an article according to the present disclosure;

FIG. 2A is an embodiment of an exploded cross-sectional side view taken along line 2A-2A of FIG. 2;

FIG. 2B is an expanded view of the indicated area of FIG. 2;

FIG. 3 is a perspective view of an embodiment of personal hygiene article incorporating an article of the present disclosure, in which the article is useful as a disposal tape;

FIG. 3A is an expanded view of the indicated area in FIG. 3;

FIG. 3B is a perspective view of the personal hygiene article shown in FIG. 3 rolled up and ready for disposal;

FIG. 4 is a side, cross-section view of a roll of tape including an embodiment of the article of the present disclosure; and

FIG. 5 is a photograph of an embodiment of a three-layer construction useful in various embodiments of the article of the present disclosure.

### DETAILED DESCRIPTION

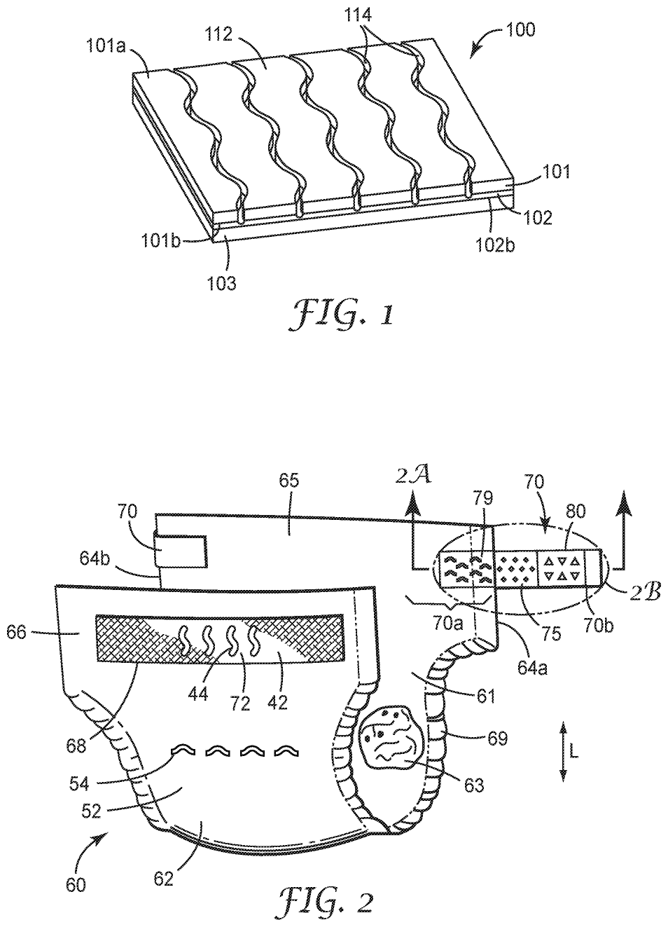

FIG. 1 is a perspective view of a multilayer construction 100 useful in the article of the present disclosure. First layer 101 is a see-through, thermoplastic film having a first color. That is, the first layer is not colorless and has a color other than white. The first layer has a first major surface 101a and second major surface 101b. The second layer 102 of the multilayer construction 100 is adjacent the second major surface 101b of the first layer 101. The second layer 102 comprises a microporous film that has an opaque, microporous region 112 and a repeating series of nonporous regions 114. The nonporous regions 114 typically correspond to regions of the multilayer film having a smaller “z” dimension (that is, lower thickness) in comparison to the opaque, microporous regions 112. In some embodiments, including the embodiment illustrated in FIG. 1, the multilayer construction 100 useful in the article of the present disclosure further comprises a third layer 103 adjacent a major surface 102b of the second layer 102 opposite the first layer 101. The third layer 103 has a second color different from the first color. That is the third layer 103 is also not colorless and has a color other than white. When the opaque, microporous region 112 and the nonporous regions 114 are seen through the first layer 101 they create an appearance on the outer surface of the article of two different colors. Since the nonporous regions 114 are in a repeating pattern in the illustrated embodiment, when seen through the first layer they create the appearance on the outer surface of a pattern of two different colors. In embodiments in which the opaque, microporous region 112 of the second layer 102 is white, the combination of the opaque, microporous region 112 in the second layer 102 and the portion of the first layer 101 overlying this region will appear as an opaque shade of the color provided in the first layer 101. In embodiments in which the nonporous region 114 of the second layer 102 is colorless, in the nonporous region 114, the first layer 101, second layer 102, and third layer 103 together appear to have a color that is a combination of the first color and the second color.

While FIG. 1 illustrates the third layer 103, the third layer need not be present in the article of the present disclosure. When the third layer is absent from the multilayer construction 100, the opaque, microporous region 112 and the nonporous region 114 seen through the first layer 101 create an appearance on the outer surface of two different shades of the same color. Since the nonporous regions 114 are in a repeating pattern in the illustrated embodiment, when seen through the first layer they create the appearance on the outer surface of a pattern of two different shades of the same color.

The size of any individual nonporous region in the article according to the present disclosure may be at least 0.3 mm², 0.4 mm², 0.5 mm², or 0.7 mm². Generally, if the color contrast between the two different colors or two different shades of the same color is relatively large, smaller individual nonporous areas (e.g., 0.3 mm² to 0.6 mm²) may be easily visible to the naked eye. However, if the color contrast between the two different colors or two different shades of the same color is relatively small, it may be desirable to have larger individual nonporous areas (e.g., larger than 0.6 mm²).

A multilayer construction such as that shown in FIG. 1 can be made in various ways. The first and second layer are thermoplastic and can be extruded separately and then laminated together (e.g., by extrusion lamination or adhesive bonding) or can be made by coextrusion. A multilayer film of at least first and second layers can be coextruded using any suitable type of coextrusion die and any suitable method of film making such as blown film extrusion or cast film extrusion. In some embodiments, a multilayer melt stream can be formed by a multilayer feedblock, such as that shown in U.S. Pat. No. 4,839,131 (Cloeren). For the best performance in coextrusion, the polymeric compositions for each layer can be chosen to have similar properties such as melt viscosity. In some embodiments, a second layer including a beta-nucleating agent or diluent as described below in a polymeric composition can be coextruded with a similar polymeric composition, lacking such an agent, which forms the first layer. The first polymeric composition for making the first layer includes a colorant (e.g., a pigment or a dye). Techniques of coextrusion are found in many polymer processing references, including Progelhof, R. C., and Throne, J. L., “Polymer Engineering Principles”, Hanser/Gardner Publications, Inc., Cincinnati, Ohio, 1993. The third layer may also be thermoplastic layer, and multilayer films including the first, second, and third layers can also be formed by coextrusion.

In some embodiments, the third layer comprises other materials such as woven webs, non-woven webs (e.g., spunbond webs, spunlaced webs, airlaid webs, meltblown web, and bonded carded webs), textiles, plastic films, and combinations thereof. The third layer can be joined to the second layer by extrusion lamination, adhesives (e.g., pressure sensitive adhesives), or other bonding methods (e.g., ultrasonic bonding, compression bonding, or surface bonding). The third layer also includes a colorant such as a pigment or dye. The third layer may also be metalized.

Various methods are useful for making the second layer including the microporous thermoplastic film disclosed herein. In some embodiments, the porosity in the microporous thermoplastic film, results from beta-nucleation. Thermoplastics (e.g., semi-crystalline polyolefins) can have more than one kind of crystal structure. For example, isotactic polypropylene is known to crystallize into at least three different forms: alpha (monoclinic), beta (pseudohexangonal), and gamma (triclinic) forms. In melt-crystallized material the predominant form is the alpha or monoclinic form. The beta form generally occurs at levels of only a few percent unless certain heterogeneous nuclei are present, or the crystallization has occurred in a temperature gradient or in the presence of shearing forces. The heterogeneous nuclei are typically known as beta-nucleating agents, which act as foreign bodies in a crystallizable polymer melt. When the polymer cools below its crystallization temperature (e.g., a temperature in a range from 60° C. to 120° C. or 90° C. to 120° C.), the loose coiled polymer chains orient themselves around the beta-nucleating agent to form beta-phase regions. The beta form of polypropylene is a meta-stable form, which can be converted to the more stable alpha form by thermal treatment and/or applying stress. Micropores can be formed in various amounts when the beta-form of polypropylene is stretched under certain conditions; see, e.g., Chu et al., “Microvoid formation process during the plastic deformation of β-form polypropylene”, Polymer, Vol. 35, No. 16, pp. 3442-3448, 1994, and Chu et al., “Crystal transformation and micropore formation during uniaxial drawing of β-form polypropylene film”, Polymer, Vol. 36, No. 13, pp. 2523-2530, 1995. Pore sizes achieved from this method can range from about 0.05 micrometer to about 1 micrometer, in some embodiments, about 0.1 micrometer to about 0.5 micrometer.

Generally, when the porosity in the microporous thermoplastic film is generated from a beta-nucleating agent, the film comprises a semi-crystalline polyolefin. Various polyolefins may be useful. Typically, the semi-crystalline polyolefin comprises polypropylene. It should be understood that a semi-crystalline polyolefin comprising polypropylene may be a polypropylene homopolymer or a copolymer containing propylene repeating units. The copolymer may be a copolymer of propylene and at least one other olefin (e.g., ethylene or an alpha-olefin having from 4 to 12 or 4 to 8 carbon atoms). Copolymers of ethylene, propylene and/or butylenes may be useful. In some embodiments, the copolymer contains up to 90, 80, 70, 60, or 50 percent by weight of polypropylene. In some embodiments, the copolymer contains up to 50, 40, 30, 20, or 10 percent by weight of at least one of polyethylene or an alpha-olefin. The semi-crystalline polyolefin may also be part of a blend of thermoplastic polymers that includes polypropylene. Suitable thermoplastic polymers include crystallizable polymers that are typically melt processable under conventional processing conditions. That is, on heating, they will typically soften and/or melt to permit processing in conventional equipment, such as an extruder, to form a sheet. Crystallizable polymers, upon cooling their melt under controlled conditions, spontaneously form geometrically regular and ordered chemical structures. Examples of suitable crystallizable thermoplastic polymers include addition polymers, such as polyolefins. Useful polyolefins include polymers of ethylene (e.g., high density polyethylene, low density polyethylene, or linear low density polyethylene), an alpha-olefin (e.g, 1-butene, 1-hexene, or 1-octene), styrene, and copolymers of two or more such olefins. The semi-crystalline polyolefin may comprise mixtures of stereo-isomers of such polymers, e.g., mixtures of isotactic polypropylene and atactic polypropylene or of isotactic polystyrene and atactic polystyrene. In some embodiments, the semi-crystalline polyolefin blend contains up to 90, 80, 70, 60, or 50 percent by weight of polypropylene. In some embodiments, the blend contains up to 50, 40, 30, 20, or 10 percent by weight of at least one of polyethylene or an alpha-olefin.

In some embodiments, the microporous thermoplastic film is made from a polymeric composition comprising a semi-crystalline polyolefin and having a melt flow rate in a range from 0.1 to 10 decigrams per minute, for example, 0.25 to 2.5 decigrams per minute.

When the porosity in the microporous thermoplastic film is generated from a beta-nucleating agent, the beta-nucleating agent may be any inorganic or organic nucleating agent that can produce beta-spherulites in a melt-formed sheet comprising polyolefin. Useful beta-nucleating agents include gamma quinacridone, an aluminum salt of quinizarin sulphonic acid, dihydroquinoacridin-dione and quinacridin-tetrone, triphenenol ditriazine, calcium silicate, dicarboxylic acids (e.g., suberic, pimelic, ortho-phthalic, isophthalic, and terephthalic acid), sodium salts of these dicarboxylic acids, salts of these dicarboxylic acids and the metals of Group IIA of the periodic table (e.g., calcium, magnesium, or barium), delta-quinacridone, diamides of adipic or suberic acids, different types of indigosol and cibantine organic pigments, quiancridone quinone, N′,N′-dicyclohexil-2,6-naphthalene dicarboxamide (available, for example, under the trade designation “NJ-Star NU-100” from New Japan Chemical Co. Ltd.), antraquinone red, and bis-azo yellow pigments. The properties of the extruded film are dependent on the selection of the beta nucleating agent and the concentration of the beta-nucleating agent. In some embodiments, the beta-nucleating agent is selected from the group consisting of gamma-quinacridone, a calcium salt of suberic acid, a calcium salt of pimelic acid and calcium and barium salts of polycarboxylic acids. In some embodiments, the beta-nucleating agent is quinacridone colorant Permanent Red E3B, which is also referred to as Q-dye. In some embodiments, the beta-nucleating agent is formed by mixing an organic dicarboxylic acid (e.g., pimelic acid, azelaic acid, o-phthalic acid, terephthalic acid, and isophthalic acid) and an oxide, hydroxide, or acid salt of a Group II metal (e.g., magnesium, calcium, strontium, and barium). So-called two component initiators include calcium carbonate combined with any of the organic dicarboxylic acids listed above and calcium stearate combined with pimelic acid. In some embodiments, the beta-nucleating agent is aromatic tri-carboxamide as described in U.S. Pat. No. 7,423,088 (Mader et al.).

The beta-nucleating agent serves the important functions of inducing crystallization of the polymer from the molten state and enhancing the initiation of polymer crystallization sites so as to speed up the crystallization of the polymer. Thus, the nucleating agent may be a solid at the crystallization temperature of the polymer. Because the nucleating agent increases the rate of crystallization of the polymer, the size of the resultant polymer particles, or spherulites, is reduced.

A convenient way of incorporating beta-nucleating agents into a thermoplastic (e.g., semi-crystalline polyolefin) useful for making a microporous film disclosed herein is through the use of a concentrate. A concentrate is typically a highly loaded, pelletized resin (e.g., polypropylene) containing a higher concentration of nucleating agent than is desired in the final microporous film. The nucleating agent is present in the concentrate in a range of 0.01% to 2.0% by weight (100 to 20,000 ppm), in some embodiments in a range of 0.02% to 1% by weight (200 to 10,000 ppm). Typical concentrates are blended with non-nucleated polyolefin, for example, in the range of 0.5% to 50% (in some embodiments, in the range of 1% to 10%) by weight of the total polyolefin content of the microporous film. The concentration range of the beta-nucleating agent in the final microporous film may be 0.0001% to 1% by weight (1 ppm to 10,000 ppm), in some embodiments, 0.0002% to 0.1% by weight (2 ppm to 1000 ppm). A concentrate can also contain other additives such as stabilizers, pigments, and processing agents.

The level of beta-spherulites in the semi-crystalline polyolefin can be determined, for example, using X-ray crystallography and Differential Scanning Calorimetry (DSC). By DSC, melting points and heats of fusion of both the alpha phase and the beta phase can be determined in a microporous film useful for practicing the present disclosure. For semi-crystalline polypropylene, the melting point of the beta phase is lower than the melting point of the alpha phase (e.g., by about 10 to 15 degrees Celsius). The ratio of the heat of fusion of the beta phase to the total heat of fusion provides a percentage of the beta-spherulites in a sample. The level of beta-spherulites can be at least 10, 20, 25, 30, 40, or 50 percent, based on the total amount of alpha and beta phase crystals in the film. These levels of beta-spherulites may be found in the film before it is stretched.

In some embodiments, the microporous thermoplastic film useful for practicing the present disclosure in any of its embodiments is formed using a thermally induced phase separation (TIPS) method. This method of making the microporous thermoplastic film typically includes melt blending a crystallizable polymer and a diluent to form a melt mixture. The melt mixture is then formed into a film and cooled to a temperature at which the polymer crystallizes, and phase separation occurs between the polymer and diluent, forming voids. In this manner a film is formed that comprises an aggregate of crystallized polymer in the diluent compound. The voided film has some degree of opacity.

In some embodiments, following formation of the crystallized polymer, the porosity of the material is increased by at least one of stretching the film in at least one direction or removing at least some of the diluent. This step results in separation of adjacent particles of polymer from one another to provide a network of interconnected micropores. This step also permanently attenuates the polymer to form fibrils, imparting strength and porosity to the film. The diluent can be removed from the material either before or after stretching. In some embodiments, the diluent is not removed. Pore sizes achieved from this method can range from about 0.2 micron to about 5 microns.

When the microporous thermoplastic film useful for practicing the present disclosure is made from a TIPS process, the film can comprise any of the semi-crystalline polyolefins described above in connection with films made by beta-nucleation. In addition, other crystallizable polymers that may be useful alone or in combination include high and low density polyethylene, poly(vinylidine fluoride), poly(methyl pentene) (e.g., poly(4-methylpentene), poly(lactic acid), poly(hydroxybutyrate), poly(ethylene-chlorotrifluoroethylene), poly(vinyl fluoride), polyvinyl chloride, poly(ethylene terephthalate), poly(butylene terephthalate), ethylene-vinyl alcohol copolymers, ethylene-vinyl acetate copolymers, polybuyltene, polyurethanes, and polyamides (e.g., nylon-6 or nylon-66). Useful diluents for providing the microporous film according to the present disclosure include mineral oil, mineral spirits, dioctylphthalate, liquid paraffins, paraffin wax, glycerin, petroleum jelly, polyethylene oxide, polypropylene oxide, polytetramethylene oxide, soft carbowax, and combinations thereof. The quantity of diluent is typically in a range from about 20 parts to 70 parts, 30 parts to 70 parts, or 50 parts to 65 parts by weight, based upon the total weight of the polymer and diluent.

Particulate cavitating agents are also useful for making microporous thermoplastic films. Such cavitating agents are incompatible or immiscible with the polymeric matrix material and form a dispersed phase within the polymeric core matrix material before extrusion and orientation of the film. When such a polymer substrate is subjected to uniaxial or biaxial stretching, a void or cavity forms around the distributed, dispersed-phase moieties, providing a film having a matrix filled with numerous cavities that provide an opaque appearance due to the scattering of light within the matrix and cavities. The microporous thermoplastic film can comprise any of the polymers described above in connection with TIPS films. The particulate cavitating agents may be inorganic or organic. Organic cavitating agents generally have a melting point that is higher than the melting point of the film matrix material. Useful organic cavitating agents include polyesters (e.g., polybutylene teraphthalate or nylon such as nylon-6), polycarbonate, acrylic resins, and ethylene norbornene copolymers. Useful inorganic cavitating agents include talc, calcium carbonate, titanium dioxide, barium sulfate, glass beads, glass bubbles (that is, hollow glass spheres), ceramic beads, ceramic bubbles, and metal particulates. The particle size of cavitating agents is such that at least a majority by weight of the particles comprise an overall mean particle diameter, for example, of from about 0.1 micron to about 5 microns, in some embodiments, from about 0.2 micron to about 2 microns. (The term “overall” refers to size in three dimensions; the term “mean” is the average.) The cavitating agent may be present in the polymer matrix in an amount of from about 2 weight percent to about 40 weight percent, about 4 weight percent to about 30 weight percent, or about 4 weight percent to about 20 weight percent, based upon the total weight of the polymer and cavitating agent. While particulate cavitating agents may be useful for making some embodiments of the microporous thermoplastic film disclosed herein, generally microporous films made with such cavitating agents provide see-through regions that are less transparent than when beta-nucleation or the TIPS process is used. Accordingly, in some embodiments, the microporous thermoplastic film comprises at least one of a beta-nucleating agent or a diluent. In some embodiments, the microporous thermoplastic film is free of a particulate cavitating agents or contains less than 2, 1.5, 1, 0.5, or 0.1 percent of such cavitating agents, based on the total weight of the film.

Additional ingredients may be included in the microporous thermoplastic film useful for practicing any of the embodiments of the present disclosure, depending on the desired application. For example, surfactants, antistatic agents, ultraviolet radiation absorbers, antioxidants, organic or inorganic colorants, stabilizers, flame retardants, fragrances, nucleating agents other than a beta-nucleating agent, and plasticizers may be included. Many of the beta-nucleating agents described above have a color. Also, colorants may be added, for example, in the form of a color concentrate or a colored master batch. In some embodiments, the microporous thermoplastic film is not colored, in other words, the opaque, microporous regions are white.

For the microporous thermoplastic films made by any of the methods described above, the film is typically stretched to form or enhance the microporous structure. Stretching the film can be carried out on a web biaxially or monoaxially. Biaxial stretching means stretching in two different directions in the plane of the backing. Typically, but not always, one direction is the machine direction or longitudinal direction “L”, and the other, different direction is the cross direction or width direction “W”. Biaxial stretching can be performed sequentially by stretching the thermoplastic backing, for example, first in one of the longitudinal or width direction and subsequently in the other of the longitudinal or width direction. Biaxial stretching can also be performed essentially simultaneously in both directions. Monoaxial stretching refers to stretching in only one direction in the plane of the backing. Typically, monoaxial stretching is performed in one of the “L” or “W” direction but other stretch directions are also possible.

In some embodiments of the article and method disclosed herein, the first layer and second layer are stretched simultaneously. In some of these embodiments, the first layer, second layer, and third layer are stretched simultaneously.

In some embodiments, the stretching increases at least one of the film's length (“L”) or width (“W”) at least 1.2 times (in some embodiments, at least 1.5, 2, or 2.5 times). In some embodiments, the stretching increases both of the film's length (“L”) and width (“W”) at least 1.2 times (in some embodiments, at least 1.5, 2, or 2.5 times). In some embodiments, the stretching increases at least one of the film's length (“L”) or width (“W”) up to 5 times (in some embodiments, up to 2.5 times). In some embodiments, the stretching increases both of the film's length (“L”) and width (“W”) up to 5 times (in some embodiments, up to 2.5 times). In some embodiments, the stretching increases at least one of the film's length (“L”) or width (“W”) up to 10 times (in some embodiments, up to 20 times or more). In some embodiments, the stretching increases both of the film's length (“L”) and width (“W”) up to 10 times (in some embodiments, up to 20 times or more).

In general, when a thermoplastic film is monoaxially or biaxially stretched at a temperature below the melting point of the thermoplastic material, particularly at a temperature below the line drawing temperature of the film, the thermoplastic film may stretch non-uniformly, and a clear boundary is formed between stretched and unstretched parts. This phenomenon is referred to as necking or line drawing. However, substantially the entire thermoplastic backing is stretched uniformly when it is stretched to a sufficiently high degree. The stretch ratio at which this occurs is referred to as the “natural stretch ratio” or “natural draw ratio.” Stretching above the natural stretch ratio is understood to provide significantly more uniform properties or characteristics such as thickness, tensile strength, and modulus of elasticity. For any given thermoplastic backing and stretch conditions, the natural stretch ratio is determined by factors such as the composition of the thermoplastic resin forming the thermoplastic backing, the morphology of the formed thermoplastic backing due to quenching conditions on the tool roll, for example, and temperature and rate of stretching. Furthermore, for biaxially stretched thermoplastic backings, the natural stretch ratio in one direction will be affected by the stretch conditions, including final stretch ratio, in the other direction. Thus, there may be said to be a natural stretch ratio in one direction given a fixed stretch ratio in the other, or, alternatively, there may be said to be a pair of stretch ratios (one in the first direction and one in the second direction) which result in the natural stretch ratio. The term “stretch ratio” refers to ratio of a linear dimension of a given portion of the thermoplastic backing after stretching to the linear dimension of the same portion before stretching. The natural stretch ratio of the most common crystalline form of polypropylene, the alpha form, has been reported to be about 6:1.

Stretching the film useful for practicing the present disclosure (in some embodiments, the multilayer film including first, second, and optionally third layers) can be carried out in a variety of ways. When the film is a web of indefinite length, for example, monoaxial stretching in the machine direction can be performed by propelling the film over rolls of increasing speed. The term “machine direction” (MD) as used herein denotes the direction of a running, continuous web of the film. A versatile stretching method that allows for monoaxial, sequential biaxial, and simultaneous biaxial stretching of the film employs a flat film tenter apparatus. Such an apparatus grasps the thermoplastic web using a plurality of clips, grippers, or other film edge-grasping means along opposing edges of the film in such a way that monoaxial, sequential biaxial, or simultaneous biaxial stretching in the desired direction is obtained by propelling the grasping means at varying speeds along divergent rails. Increasing clip speed in the machine direction generally results in machine-direction stretching. Means such as diverging rails generally results in cross-direction stretching. The term “cross-direction” (CD) as used herein denotes the direction which is essentially perpendicular to the machine direction. Monoaxial and biaxial stretching can be accomplished, for example, by the methods and apparatus disclosed in U.S. Pat. No. 7,897,078 (Petersen et al.) and the references cited therein. Flat film tenter stretching apparatuses are commercially available, for example, from Brückner Maschinenbau GmbH, Siegsdorf, Germany.

Stretching the film is typically performed at elevated temperatures, for example, up to 150° C. Heating the film may allow it to be more flexible for stretching. Heating can be provided, for example, by IR irradiation, hot air treatment or by performing the stretching in a heat chamber. In some embodiments of the mechanical fastener according to the present disclosure, heating is only applied to a second surface of the film opposite the first surface from which the mechanical fastening elements project to minimize any damage to the mechanical fastening elements that may result from heating. For example, in these embodiments, only rollers that are in contact with the second surface of the film are heated. In some embodiments, stretching the film is carried out at a temperature range from 50° C. to 130° C.

In the article according to the present disclosure, the construction of the first layer, second layer, and optionally third layer may have a variety of thicknesses. For example, the initial thickness (i.e., before any stretching) of the multilayer film may be up to about 750, 500, 400, 250, or 150 micrometers, depending on the desired application. In some embodiments, the initial thickness of the film is at least about 50, 75, or 100 micrometers, depending on the desired application. In some embodiments, the initial thickness of the film is in a range from 50 to about 225 micrometers, from about 75 to about 200 micrometers, or from about 100 to about 150 micrometers. The film may have an essentially uniform cross-section, or, in the case of mechanical fasteners, the film may have structure beyond what is provided by the upstanding posts, which may be imparted, for example, by at least one of the forming rolls described below.

In some embodiments, stretching a film described above in order to form or enhance microporosity provides an increase in opacity of at least 10, 15, 20, 25, or 30 percent. The increase in opacity may be, for example, up to 90, 85, 80, 75, 70, 65, 60, 55, or 50 percent. The initial opacity is affected, for example, by the thickness of the film. Stretching a film typically results in a decrease in thickness, which would typically lead to a decrease in opacity. However, stress whitening and micropore formation leads to an increase in opacity. For the purposes of the present disclosure, opacity can be measured using a spectrophotometer with the “L” value measured separately against a black background and against a white background, respectively. The opacity is calculated as (L measured against the black background/L measured against the white background) times 100. The “L” value is one of three standard parameters in the CIELAB color space scale established by the International Commission on Illumination. “L” is a brightness value, ranging from 0 (black) to 100 (highest intensity). A percentage change in opacity that results from stretching is calculated by [(opacity after stretching−opacity before stretching)/opacity before stretching] times 100.

In some embodiments, stretching a film described above in order to form or enhance microporosity provides a decrease in the grayscale value of the film of at least twenty percent. In some embodiments, stretching provides a decrease in a grayscale value of at least 25, 30, 40, or 50 percent. The decrease in grayscale value may be, for example, up to 90, 85, 80, 75, 70, 65, or 60 percent. For the purposes of this disclosure, the grayscale value is measured in transmission mode using the method described in the Example section, below. Stretching a film typically results in a decrease in thickness, which would typically lead to an increase in the grayscale value measured in transmission mode. However, stress whitening and micropore formation leads to decrease in transmission mode grayscale values. A percentage change in grayscale value that results from stretching the film is calculated by [(grayscale value after stretching−grayscale value before stretching)/grayscale value before stretching] times 100. In some embodiments, the microporous film has a grayscale value of up to 40 (in some embodiments, up to 35, 30, 25, 20 or 15). In some embodiments, the grayscale values for the microporous films disclosed herein are comparable or better than those achieved for polyolefin films of similar composition but incorporating conventional amounts of IR blocking agents such as titanium dioxide.

The opacity and grayscale measurement of the microporous film relate to its ability to transmit light. As used herein, the term “light” refers to electromagnetic radiation, whether visible to the unaided human eye or not. Ultraviolet light is light having a wavelength in a range from about 250 nanometers (nm) to 380 nm. Visible light is light having a wavelength in a range from 380 nanometers (nm) to 700 nm. Infrared light has a wavelength in a range from about 700 nm to 300 micrometers. After the microporous film useful for practicing the present disclosure has been stretched, it has decreased transmission to ultraviolet, visible, and infrared light. The micropores in the stretched film tend to scatter light in the ultraviolet, visible, and infrared ranges.

Referring again to FIG. 1, the nonporous regions 114 may be made by a number of useful methods. For example, a nip made from two heated rolls in which at least one of the rolls has raised areas in the shape of the nonporous regions 114 may be useful. The heat and pressure in the nip can collapse the microporous structure in the raised areas to form the nonporous regions. The illustrated embodiment can be made if only one roll includes the raised areas, and the other roll has a smooth surface such that the third layer 103 has a smooth surface. However, if both rolls have raised areas in the shape of the nonporous regions 114, third layer 103 may also include depressions (not shown).

Heat, pressure, or a combination thereof may be useful for providing the nonporous regions. Typically, the nonporous region is heated to the melting temperature of the thermoplastic in the microporous film. Melting the microporous film in the nonporous region results in a permanent change in the structure of the film in the nonporous region, which can be accompanied by some film shrinkage in that region. Heating can be carried out in a press or a heated nip having a raised image of the nonporous region so that pressure accompanies the heating to collapse the microporous structure. Pressure alone may provide a temporary change in the microporous structure of the microporous film in some instances. When using a static press, it can be useful to use a rubber surface on the film side opposite the side that is exposed to the raised and heated image. The rubber surface can prevent two hard surfaces from forming a hole in the film while the nonporous region is being made. In a nip, the pressure and gap can be adjusted as well as the line speed to prevent forming holes in the film.

Heating may also be carried out with hot air or with a directed radiation source such as a laser. A variety of different types of laser may be useful. For example, a carbon dioxide laser may be useful. An ultraviolet laser and diode laser may also be useful. Suitable wavelengths for the laser can in a range from 200 nm to 11,000 nm. The laser wavelength and absorption properties of the material can be selected to be matched or nearly matched so as to create the heating of material. For a person skilled in the art, the suitable power for the laser, beam size on the material, and speed of the beam movement across the material can be adjusted to achieve the desired heating. This matching of laser and material can be advantageous, for example, when the microporous thermoplastic film is a layer with a multilayer construction. Heating with the laser can be adjusted to a location of the microporous film within the multilayer construction (e.g., multilayer film). The heating can be made in a pattern by directing the radiation across the surface to expose an area of material, or the radiation can be directed across the surface of a suitable mask so that a patterned area is exposed to the radiation. The microporous film may be positioned outside of the focal plane of the laser to adjust the level of heating.

For some applications such as heat seal films, recording media, and oil-absorbing cosmetic sheets, it has been shown that changing the microporous structure in a region of a microporous film can change the opacity in that region. See, for example, GB 2323327, published Sep. 23, 1998, GB 2252838, published Aug. 19, 1992, and U.S. Pat. App. Pub. No. 2003/091618 (Seth et al.). However, in some of these cases, the change is provided in a random fashion, for example, by an impact during the use of the film that cannot provide a predetermined pattern or image. A change in the microporous structure by impact may also not be permanent. In other cases, the change is only provided along the edge of a film and therefore does not provide a nonporous region within an opaque, microporous region.

In some embodiments, the article of the present disclosure is a mechanical fastener. In some embodiments, the mechanical fastening elements of the mechanical fastener are male fastening elements. In some of these embodiments, the male fastening elements comprise upstanding posts having bases attached to the thermoplastic first layer. The first layer and the upstanding posts are typically integral (that is, formed at the same time as a unit, unitary). The first layer is typically in the form of a sheet or web that may have an essentially uniform thickness with the upstanding posts directly attached to the thermoplastic film.

Upstanding posts on a thermoplastic film can be made, for example, by conventional extrusion through a die and cast molding techniques. In some embodiments, a thermoplastic composition is fed onto a continuously moving mold surface with cavities having the inverse shape of the upstanding posts. The thermoplastic composition can be passed between a nip formed by two rolls or a nip between a die face and roll surface, with at least one of the rolls having the cavities (i.e., at least one of the rolls is a tool roll). Pressure provided by the nip forces the composition into the cavities. In some embodiments, a vacuum can be used to evacuate the cavities for easier filling of the cavities. The nip has a gap that is typically large enough such that a coherent film is formed over the cavities. The mold surface and cavities can optionally be air or water cooled before stripping the integrally formed film and upstanding posts from the mold surface such as by a stripper roll.

Suitable tool rolls can be made, for example, by forming (e.g., by computer numerical control with drilling, photo etching, using galvanic printed sleeves, laser drilling, electron beam drilling, metal punching, direct machining, or lost wax processing) a series of holes having the inverse shape of the upstanding posts into the cylindrical face of a metal mold or sleeve. Other suitable tool rolls include those formed from a series of plates defining a plurality of post-forming cavities about its periphery such as those described, for example, in U.S. Pat. No. 4,775,310 (Fischer). Cavities may be formed in the plates by drilling or photoresist technology, for example. Other suitable tool rolls may include wire-wrapped rolls, which are disclosed along with their method of manufacturing, for example, in U.S. Pat. No. 6,190,594 (Gorman et al.). Another example of a method for forming a thermoplastic backing with upstanding posts includes using a flexible mold belt defining an array of upstanding post-shaped cavities as described in U.S. Pat. No. 7,214,334 (Jens et al.). Yet other useful methods for forming a thermoplastic backing with upstanding posts can be found in U.S. Pat. No. 6,287,665 (Hammer), U.S. Pat. No. 7,198,743 (Tuma), and U.S. Pat. No. 6,627,133 (Tuma).

The upstanding posts, which may be made, for example, by any of the methods described above, may have a shape that tapers, for example, from base portion attached to the film to a distal tip. The base portion may have a larger width dimension than the distal tip, which may facilitate the removal of the post from the mold surface in the methods described above.

The male fastening elements in the mechanical fastener disclosed herein may have loop-engaging heads that have an overhang or may be upstanding posts having distal tips that can be formed into loop-engaging heads, if desired. The term “loop-engaging” as used herein relates to the ability of a male fastening element to be mechanically attached to a loop material. Generally, male fastening elements with loop-engaging heads have a head shape that is different from the shape of the post. For example, the male fastening element may be in the shape of a mushroom (e.g., with a circular or oval head enlarged with respect to the stem), a hook, a palm-tree, a nail, a T, or a J (e.g., as shown and described in U.S. Pat. No. 5,953,797 (Provost et al.). The loop-engageability of male fastening elements may be determined and defined by using standard woven, nonwoven, or knit materials. A region of male fastening elements with loop-engaging heads generally will provide, in combination with a loop material, at least one of a higher peel strength, higher dynamic shear strength, or higher dynamic friction than a region of posts without loop-engaging heads. Typically, male fastening elements that have loop-engaging heads have a maximum thickness dimension (in either dimension normal to the height) of up to about 1 (in some embodiments, 0.9, 0.8, 0.7, 0.6, 0.5, or 0.45) millimeter.

In some embodiments, the distal tips of the upstanding posts that are formed according to any of the above methods are deformed to form caps with loop-engaging overhangs. A combination heat and pressure, sequentially or simultaneously, may be used to deform the distal tips of the posts to form caps. In some embodiments, deforming comprises contacting the distal tips with a heated surface. The heated surface may be a flat surface or a textured surface such as that disclosed in U.S. Pat. No. 6,708,378 (Parellada et al.) or U.S. Pat. No. 5,868,987 (Kampfer et al.). In some embodiments, wherein the film with upstanding posts is a web of indefinite length, the deforming comprises moving the web in a first direction through a nip having a heated surface member and an opposing surface member such that the heated surface member contacts the distal tips. In these embodiments, the heated surface may be, for example, a capping roll. In some embodiments, the surface used to contact the distal tips is not heated. In these embodiments, the deformation is carried out with pressure and without heating. In some embodiments, the heated surface may be a heated roll opposite a curved support surface forming a variable nip having a variable nip length as described, for example, in U.S. Pat. No. 6,368,097 (Miller et al.). The curved support surface may curve in the direction of the heated roll, and the heated roll may include a feeding mechanism for feeding the film with upstanding posts through the variable nip to compressively engage the web between the heated roll and the support surface.

Another suitable method for forming a film with upstanding posts attached to a thermoplastic film backing is profile extrusion, which is described, for example, in U.S. Pat. No. 4,894,060 (Nestegard). In this method a flow stream of a thermoplastic composition is passed through a patterned die lip (e.g., cut by electron discharge machining) to form a web having downweb ridges. The ridges are then transversely sliced at spaced locations along the extension of the ridges to form upstanding posts with a small separation caused by the cutting blade. It should be understood that “upstanding posts” do not include such ridges before they are cut. However, the patterned die lip may be considered a tool to provide a film having upstanding posts on a backing. The separation between the upstanding posts is then increased by stretching the film in the direction of the ridges using one of the stretching methods described above. The ridges themselves would also not be considered “loop-engaging” because they would not be able to engage loops before they are cut and stretched. In some embodiments, methods according to the present disclosure do not include cutting ribs (e.g., made by profile extrusion).

In addition to the continuous methods described above, it is also envisioned that films with upstanding posts can be prepared using batch processes (e.g., single piece injection molding). The film may have any suitable dimension, but length (L) and width (W) dimensions of at least 10 cm may be useful.

The upstanding posts, in any of the embodiments of the mechanical fastener including male fastening elements disclosed herein, which may be made, for example, by any of the methods described above, may have a variety of cross-sectional shapes. For example, the cross-sectional shape of the post may be a polygon (e.g., square, rectangle, hexagon, or pentagon), which may be a regular polygon or not, or the cross-sectional shape of the post may be curved (e.g., round or elliptical).

In some embodiments, the upstanding posts have a maximum height (above the film) of up to 3 millimeters (mm), 1.5 mm, 1 mm, or 0.5 mm and, in some embodiments, a minimum height of at least 0.05 mm, 0.075 mm, 0.1 mm, or 0.2 mm. In some embodiments, the posts have aspect ratio (that is, a ratio of height over a width dimension) of at least about 2:1, 3:1, or 4:1. The aspect ratio may be, in some embodiments, up to 10:1. For posts with caps, the caps are typically larger in area than the cross-sectional area of the posts. A ratio of a width dimension of the cap to the post measured just below the cap is typically at least 1.5:1 or 3:1 and may be up to 5:1 or greater. The capped posts are typically shorter than the posts before capping. In some embodiments, the capped posts have a height (above the film) of at least 0.025 mm, 0.05 mm, or 0.1 mm and, in some embodiments, up to 2 mm, 1.5 mm, 1 mm, or 0.5 mm. The posts, which may be capped or not, may have a cross-section with a maximum width dimension of up to 1 (in some embodiments, up to 0.75, 0.5, or 0.45) mm. In some embodiments, the posts have a cross-section with a width dimension between 10 μm and 250 μm. The term “width dimension” should be understood to include the diameter of a post with a circular cross-section. When the post has more than one width dimension (e.g., in a rectangular or elliptical cross-section shaped post or a post that tapers as described above), the aspect ratio described herein is the height over the largest width dimension.

The upstanding posts are typically spaced apart on the backing. The term “spaced-apart” refers to posts that are formed to have a distance between them. The bases of “spaced-apart” posts, where they are attached to the film, do not touch each other before or after stretching the film when the film is in an unbent configuration. In the mechanical fastener according to and/or made according to the present disclosure, the spaced-apart upstanding posts have an initial density (i.e., before any stretching of the film) of at least 10 per square centimeter (cm²) (63 per square inch in²). For example, the initial density of the posts may be at least 100/cm² (635/in²), 248/cm² (1600/in²), 394/cm² (2500/in²), or 550/cm² (3500/in²). In some embodiments, the initial density of the posts may be up to 1575/cm² (10000/in²), up to about 1182/cm² (7500/in²), or up to about 787/cm² (5000/in²). Initial densities in a range from 10/cm² (63/in²) to 1575/cm² (10000/in²) or 100/cm² (635/in²) to 1182/cm² (7500/in²) may be useful, for example. The spacing of the upstanding posts need not be uniform. After stretching the density of the upstanding posts is less than the initial density of the upstanding posts. In some embodiments, the upstanding posts have a density after stretching of at least 2 per square centimeter (cm²) (13 per square inch in²). For example, the density of the posts after stretching may be at least 62/cm² (400/in²), 124/cm² (800/in²), 248/cm² (1600/in²), or 394/cm² (2500/in²). In some embodiments, the density of the posts after stretching may be up to about 1182/cm² (7500/in²) or up to about 787/cm² (5000/in²). Densities after stretching in a range from 2/cm² (13/in²) to 1182/cm² (7500/in²) or 124/cm² (800/in²) to 787/cm² (5000/in²) may be useful, for example. Again, the spacing of the posts need not be uniform.

Mechanical fasteners, which are also called hook and loop fasteners, typically include a plurality of closely spaced upstanding projections with loop-engaging heads useful as hook members, and loop members typically include a plurality of woven, nonwoven, or knitted loops. Mechanical fasteners are widely used, for example, in personal hygiene articles (that is, wearable disposable absorbent articles) to fasten such articles around the body of a person. In typical configurations, a hook strip or patch on a fastening tab attached to the rear waist portion of a diaper or incontinence garment, for example, can fasten to a landing zone of loop material on the front waist region, or the hook strip or patch can fasten to the backsheet (e.g., nonwoven backsheet) of the diaper or incontinence garment in the front waist region. However, mechanical fasteners are useful for providing releasable attachment in numerous applications (e.g., abrasive discs, assembly of automobile parts, as well as personal hygiene articles).

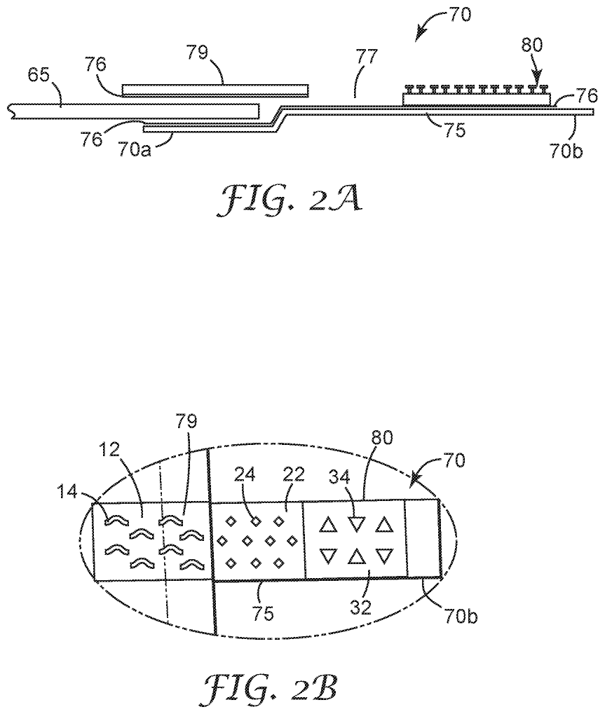

FIG. 2 is a perspective view of an embodiment of a personal hygiene article incorporating an article according to the present disclosure. The personal hygiene article is a diaper 60 having an essentially hourglass shape. The diaper comprises an absorbent core 63 between a liquid permeable top sheet 61 that contacts the wearer's skin and an outwardly facing liquid impermeable back sheet 62. Diaper 60 has a rear waist region 65 having two fastening tabs 70 arranged at the two longitudinal edges 64a, 64b of diaper 60. The diaper 60 may comprise an elastic material 69 along at least a portion of longitudinal edges 64a and 64b to provide leg cuffs. When attaching the diaper 60 to a wearer's body, the user's ends 70b of fastening tabs 70 can be attached to a target area 68 comprising fibrous material 72 arranged on the backsheet 62 of the front waist region 66. The longitudinal direction “L” of the personal hygiene article (e.g., diaper 60) refers to the direction that the article extends from the front to rear of the user. Therefore, the longitudinal direction refers to the length of the personal hygiene article between the rear waist region 65 and the front waist region 66. The lateral direction of the personal hygiene article (e.g., diaper 60) refers to the direction that the article extends from the left side to the right side (or vice versa) of the user (i.e., from longitudinal edge 64a to longitudinal edge 64b in the embodiment of FIG. 2).

An exemplary cross-section of the fastening tab 70 taken through line 2A-2A in FIG. 2 is shown in FIG. 2A. Fastening tab 70 has a manufacturer's end 70a secured to the diaper rear waist region 65 and a user's end 70b that includes the fastening portion. The manufacturer's end 70a corresponds to the part of fastening tab 70 which is fixed or secured to the diaper 60 during the manufacture of the diaper 60. The user's end is typically gripped by the user when attaching the diaper 60 to the wearer and is typically not fixed to the diaper during manufacturing. Fastening tab 70 usually extends beyond longitudinal edges 64a, 64b of the diaper 60.

In the embodiment illustrated in FIG. 2A, fastening tab 70 comprises a tape backing 75 bearing adhesive 76. Adhesive 76 joins optional mechanical fastener 80 to the tape backing 75 and joins the tape backing 75 to the rear waist region 65 of the diaper. In the illustrated embodiment, exposed adhesive 77 may be present between the mechanical fastener 80 and the diaper rear waist region 65. Fastening tab 70 further comprises release tape 79 to contact the exposed part of adhesive 77 when the user's end 70b is folded onto diaper rear waist region 65 (e.g., during packaging and shipping of diaper 60 as shown for the fastening tab 70 at longitudinal edge 64b). As shown in FIG. 2A, the release tape 79 is attached to the tape backing 75 (in some embodiments, directly attached as shown) along only one of its edges, leaving the opposite edge to be joined to the diaper rear waist region 65 during the manufacture of the personal hygiene article. The release tape 79 therefore is generally understood in the art to be permanently attached to the fastening tab 70 and ultimately to the personal hygiene article. In this way, release tape 79 is understood to be different from a release liner that is temporarily placed over exposed adhesive and discarded when the adhesive is in use. The release tape 79 may be joined to the tape backing 75 and diaper rear waist region 65 using adhesive 76 although in some embodiments, thermobonding, ultrasonic bonding, or laser bonding may be useful. Other configurations of release tape 79 are possible depending on the configuration of the attachment of the fastening tab 70 to diaper 60. The tape backing 75 at the user's end 70b of the fastening tab 70 may exceed the extension of the adhesive 76 and optional mechanical fastener 80 thereby providing a fingerlift.

In some embodiments, when a fastening tab is manufactured, the release tape 79 is folded back on itself and can be applied to the tape backing 75 in a pre-folded condition although it is possible in some cases to fold the release tape 79 after attaching one end to the tape backing. The release tape 79 may also be attached to the tape backing 75 using a separate strip or patch (not shown). The strip or patch can be made from a material such as any of the films and fibrous materials described herein below. When the release tape 79 is coated with an adhesive layer on a surface opposite the release surface, the strip or patch can adhere to both the release tape 79 and the tape backing 75 to connect them. Otherwise, other bonding methods (e.g., ultrasonic bonding) may be used.

FIG. 2 illustrates a variety of embodiments of the article according to the present disclosure in the same diaper 60. As illustrated in FIG. 2 and the expanded view of the fastening tab 70 shown in FIG. 2B, release tape 79 includes a first layer that has a first major surface that is visible in FIG. 2. When seen through the first layer, opaque, microporous region 12 and nonporous region 14 appear as two different colors or two different shades of the same color. Also, in the illustrated embodiment, tape backing 75 is includes a first layer that has a first major surface that is visible in FIG. 2. When seen through the first layer opaque, microporous region 22 and nonporous region 24 appear as two different colors or two different shades of the same color. Furthermore, mechanical fastener 80 includes a first layer that has a first major surface that is visible in FIG. 2. When seen through the first layer, opaque, microporous region 32 and nonporous region 34 appear as two different colors or two different shades of the same color. Target area 68 includes a mechanical fastener including a first layer that has a first major surface that is visible in FIG. 2. When seen through the first layer, opaque, microporous region 42 and nonporous region 44 appear as two different colors or two different shades of the same color. Finally, backsheet 62 includes a first layer that has a first major surface that is visible in FIG. 2. When seen through the first layer, opaque, microporous region 52 and nonporous region 54 appear as two different colors or two different shades of the same color. Although illustrated diaper 60 includes backsheet 62, release tape 79, tape backing 75, target area 68, and mechanical fastener 80 all have an opaque, microporous region and a nonporous region exhibiting two different colors or two different shades of the same color, any one of these or any combination of two of these may be present. As both target area 68 and mechanical fastener 80 can include at least nonporous region 34, 44 within the opaque, microporous region 32, 42, it should be understood that both hook and loop materials are included in the term “mechanical fasteners”.

In FIGS. 2 and 2B, each of the backsheet 62, release tape 79, tape backing 75, and mechanical fasteners 80 and 72 include a nonporous region 54, 14, 24, 34, and 44 that is included in a pattern of nonporous regions, which when seen through the first layer create the appearance on the outer surface of a pattern of two different colors or two different shades of the same color, although this is not a requirement. There may be more than one nonporous region within the opaque, microporous region that does not necessarily form a repeating pattern. For example, multiple nonporous regions in the form of alphabetical letters can be used together to form a word. The nonporous region(s) 54, 14, 24, 34, and 44 or, in some embodiments, the pattern of nonporous regions can be in the form of a number, picture, symbol, geometric shape, alphabetical letter, bar code, or any combination thereof. Any of these numbers, pictures, symbols, geometric shapes, alphabetical letters, or combination thereof may be part of a company name, logo, brand name, or trademark picture if desired.

In the article according to the present disclosure, the relative areas of the nonporous regions and the opaque, microporous region may be different in different embodiments. The nonporous region can make up at least 5, 10, 20, 25, 50, 75, or 90 percent of the visible area of the backsheet, tape backing, release tape, or mechanical fastener. For some patterns (e.g., a pattern of rhombuses or other geometric shapes), the opaque, microporous region may appear as strands separating the nonporous regions. For other patterns, the nonporous regions may appear more widely separated on a continuous, opaque, microporous background.

In some embodiments of the article disclosed herein (e.g., in the target area 68 shown in FIG. 2) the article includes female fastening elements, for example, loops disposed on the first major surface of the first layer. The loops may be part of a fibrous structure formed by any of several methods such as weaving, knitting, warp knitting, weft insertion knitting, circular knitting, or methods for making nonwoven structures. In some embodiments, the loops are included in a nonwoven web or a knitted web. The term “non-woven” refers to a material having a structure of individual fibers or threads that are interlaid but not in an identifiable manner such as in a knitted fabric. Examples of non-woven webs include spunbond webs, spunlaced webs, airlaid webs, meltblown web, and bonded carded webs. Useful loop materials may be made of natural fibers (e.g., wood or cotton fibers), synthetic fibers (e.g., thermoplastic fibers), or a combination of natural and synthetic fibers. Examples of suitable materials for forming thermoplastic fibers include polyolefins (e.g., polyethylene, polypropylene, polybutylene, ethylene copolymers, propylene copolymers, butylene copolymers, and copolymers and blends of these polymers), polyesters, and polyamides. The fibers may also be multi-component fibers, for example, having a core of one thermoplastic material and a sheath of another thermoplastic material.

Referring again to FIG. 2, examples of loop tapes that may suitably be applied to the target area 68 to provide an exposed fibrous material 72, are disclosed, for example, in U.S. Pat. No. 5,389,416 (Mody et al.) and U.S. Pat. No. 5,256, 231 (Gorman et al.) and EP 0,341,993 (Gorman et al.). As described in U.S. Pat. No. 5,256,231 (Gorman et al.), the fibrous layer in a loop material according to some embodiments can comprise arcuate portions projecting in the same direction from spaced anchor portions on the first major surface of the first layer of thermoplastic film. Any of the fibrous loop materials may be extrusion-bonded, adhesive-bonded, and/or sonically-bonded to the microporous film. For loop materials extrusion bonded to the film, stretching can be carried out after the extrusion bonding. Stretching the film may be carried out before or after adhesively or sonically bonding the fibrous loop material to the film.

The microporous regions in the articles according to the present disclosure provide advantages other than the color contrast between the microporous region and the nonporous region. The ability of the microporous films to block the transmission of light (e.g., by scattering) allows them to be detected in inspection systems that rely upon shining a light onto a substrate and detecting the amount of light received from the area of the irradiated substrate. For example, in the manufacture of a personal hygiene article, the presence or position of a microporous film disclosed herein or a portion thereof incorporated into the article can be detected because of its ability to block ultraviolet, visible, and/or infrared light. The response of the microporous film to irradiation by at least one of ultraviolet, visible, or infrared light is evaluated. Subsequently, during manufacturing a personal hygiene article can be irradiated, and at least one of the ultraviolet, visible, or infrared radiation received from the irradiated personal hygiene article can be detected and analyzed for the predefined response of the microporous film. The position of the microporous film can be determined using an image analyzer that can detect predefined variations in grayscale values, for example, that correspond to the positions of the microporous film and other components. The ability of the microporous film disclosed herein to scatter infrared light allows it to be detected even when it is between other layers of materials in the composite article. For more information regarding methods of detecting microporous films in a composite article, see U.S. Pat. No. 9,278,471 (Chandrasekaran et al.).

Furthermore, microporous films tend to have lower densities than their non-microporous counterparts. A low-density microporous film feels softer to the touch than films having comparable thicknesses but higher densities. The density of the film can be measured using conventional methods, for example, using helium in a pycnometer. In some embodiments, stretching a film containing beta-spherulites provides a decrease in density of at least three percent. In some embodiments, this stretching provides at decrease in density of at least 5 or 7.5 percent. For example, the stretching provides at decrease in density in a range from 3 to 15 percent or 5 to 10 percent. A percentage change in density that results from stretching the film is calculated by [(density before stretching−density after stretching)/density before stretching] times 100. The softness of the film can be measured, for example, using Gurley stiffness.

The article according to the present disclosure can be converted to any desired size and shape. The article may be in the form of a fastening tab as shown in FIGS. 2, 2A, and 2B, or the article may be attached on the ears of a personal hygiene article. Also, the mechanical fastener useful for practicing the present disclosure can be converted to any desired size and shape. For example, a personal hygiene article having ears may include a larger patch of male fastening elements relative to a mechanical fastener patch on a fastening tab. Also, a personal hygiene article can have two smaller target zones of loop material along the longitudinal edges of the back sheet instead of the large target area 68 shown in FIG. 2.

In the open configuration shown in FIG. 2A, the geometry of the tape backing 75 and the release tape 79 results in a Y-shaped bond being formed around the diaper edge in the rear waist region 65, which is often referred to in the industry as a Y-bond. However, other configurations of a release surface on a tape are possible, which tapes may or may not include a mechanical fastener. For example, a tape may be partially coated on its second surface with a release coating (e.g., a silicone, fluorochemical, or carbamate coating) and partially coated on its first surface with an adhesive. A fastening tab may be cut from such a tape and attached through its proximal end to the edge of a diaper with its release surface exposed. A distal end of the tab may be folded into a loop so that the adhesive is in contact with the release coating. Such a configuration is described in U.S. Pat. No. 3,930,502 (Tritsch). In another example, the tape may be partially coated with a release coating and partially coated with an adhesive on the same surface. A fastening tab may be cut from the tape and attached through its proximal end to the edge of a diaper with adhesive on its distal end, and the distal end of the tab may be folded back onto itself so that the adhesive is in contact with the release coating. The tape backing may be a continuous piece as shown at 75 in FIG. 2A, or when a stretchable film is desired, for example, there may be two pieces of a backing both attached to an elastic film as described in Int. Pat. Appl. Pub. No. WO 2004/075803 (Loescher et al.). Still other useful configurations of fastening tabs are described in U.S. Pat. Appl. Pub. No. 2007/0286976 (Selen et al.). In any of the embodiments of the article of the present disclosure in which the article is a release tape or includes a release surface, the article is typically provided with a release coating (e.g., a silicone, fluorochemical, or carbamate coating).

The adhesive 76 in any of the embodiments of the article according to the present disclosure is generally made up of an adhesive having a peel strength that is sufficient to permanently attach the tape backing 75 to the outside surface of an absorbent article and, in some embodiments, to permanently attach the mechanical fastener 80 to the tape backing 75. The adhesive used may be any conventional adhesive, including pressure sensitive adhesives (PSAs) and non-pressure sensitive adhesives. PSAs are well known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as PSAs are polymers designed and formulated to exhibit the requisite viscoelastic properties resulting in a desired balance of tack, peel adhesion, and shear holding power. Suitable pressure sensitive adhesives include acrylic resin and natural or synthetic rubber-based adhesives and may be hot melt pressure sensitive adhesives. Illustrative rubber-based adhesives include styrene-isoprene-styrene, styrene-butadiene-styrene, styrene-ethylene/butylene-styrene, and styrene-ethylene/propylene-styrene that may optionally contain diblock components such as styrene isoprene and styrene butadiene. The adhesive may be applied using hot-melt, solvent, or emulsion techniques.

In personal hygiene articles according to the present disclosure and/or incorporating an article according to the present disclosure, such as that shown in FIG. 2, the topsheet 61 is typically permeable to liquid and designed to contact a wearer's skin, and the outwardly facing backsheet 62 is typically impermeable to liquids. There is typically an absorbent core 63 encased between the topsheet and the backsheet. Various materials can be useful for the topsheet 61, the backsheet 62, and the absorbent core 63 in an absorbent article according to the present disclosure. Examples of materials useful for topsheets 61 include apertured plastic films, woven fabrics, nonwoven webs, porous foams, and reticulated foams. In some embodiments, the topsheet 61 is a nonwoven material. Examples of suitable nonwoven materials include spunbond or meltblown webs of fiber forming polymer filaments (e.g., polyolefin, polyester, or polyamide filaments) and bonded carded webs of natural polymers (e.g., rayon or cotton fibers) and/or synthetic polymers (e.g., polypropylene or polyester fibers). The nonwoven web can be surface treated with a surfactant or otherwise processed to impart the desired level of wettability and hydrophilicity. The backsheet 62 is sometimes referred to as the outer cover and is the farthest layer from the user. The backsheet 62 functions to prevent body exudates contained in absorbent core from wetting or soiling the wearer's clothing, bedding, or other materials contacting the diaper. The backsheet 62 can be a thermoplastic film (e.g., a poly(ethylene) film). The thermoplastic film may be embossed and/or matte finished to provide a more aesthetically pleasing appearance. The backsheet 62 can also include woven or nonwoven fibrous webs, for example, laminated to the thermoplastic films or constructed or treated to impart a desired level of liquid impermeability even in the absence of a thermoplastic film. Suitable backsheets 62 also include vapor or gas permeable microporous “breathable” materials that are substantially impermeable to liquid. Suitable absorbent cores 63 include natural, synthetic, or modified natural polymers that can absorb and hold liquids (e.g., aqueous liquids). Such polymers can be crosslinked (e.g., by physical entanglement, crystalline domains, covalent bonds, ionic complexes and associations, hydrophilic associations such as hydrogen bonding, and hydrophobic associations or Van der Waals forces) to render them water insoluble but swellable. Such absorbent materials are usually designed to quickly absorb liquids and hold them, usually without release. Examples of suitable absorbent materials useful in absorbent articles disclosed herein include wood pulp or other cellulosic materials and super absorbent polymers (SAP).

Personal hygiene articles (e.g., incontinence articles and diapers) according to the present disclosure and/or including an article disclosed herein may have any desired shape such as a rectangular shape, a shape like the letter I, a shape like the letter T, or an hourglass shape. The personal hygiene article may also be a refastenable pants-style diaper with fastening tabs 70 along each longitudinal edge. In some embodiments, including the embodiment shown in FIG. 2, the topsheet 61 and backsheet 62 are attached to each other and together form chassis all the way out to the first and second longitudinal opposing edges 64a and 64b. In some embodiments, only one of the topsheet 61 or the backsheet 62 extends to the first and second longitudinal opposing edges 64a and 64b. In other embodiments, the chassis can include separate side panels that are attached to the sandwich of at least topsheet 61, backsheet 62, and absorbent core 63 during manufacturing of the absorbent article, for example, to form ear portions. The side panels can be made of a material that is the same as the topsheet 61 or backsheet 62 or may be made from a different material (e.g., a different nonwoven). In these embodiments, the side panels also form part of the chassis.

The personal hygiene article according to the present disclosure also includes sanitary napkins. A sanitary napkin typically includes a backsheet that is intended to be placed adjacent to the wearer's undergarment. Adhesive or mechanical fasteners are provided on the backsheet to attach the sanitary napkin to the wearer's undergarment. The sanitary napkin typically also includes a topsheet and absorbent core. The backsheet, topsheet, and absorbent core can be made from any of the materials described above for these components in diapers or incontinence articles. The sanitary napkin may have any desired shape such as an hourglass, keyhole, or generally rectangular shape. The backsheet may also include flaps that are intended to wrap around to the opposite side of the wearer's undergarment. The backsheet includes a first layer of a colored, see-through thermoplastic film and a second layer that includes an opaque, microporous region and a nonporous region, wherein when seen through the first layer, the opaque, microporous region and nonporous region appear as two different colors or two different shades of the same color. The nonporous region or, in some embodiments, the pattern of nonporous regions can be in the form of a number, picture, symbol, geometric shape, alphabetical letter, bar code, or any combination thereof. Any of these numbers, pictures, symbols, geometric shapes, alphabetical letters, or combination thereof may be part of a company name, logo, brand name, or trademark picture if desired.

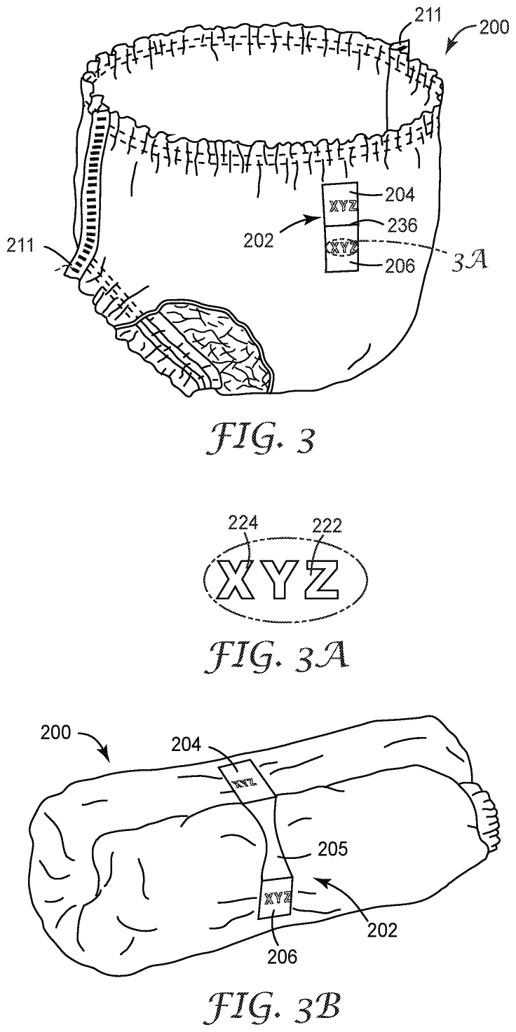

Another embodiment of an article according to the present disclosure is shown in FIGS. 3, 3A, and 3B in connection with a pants or shorts style incontinence article 200, which may be an infant diaper or adult incontinence article. After use of such a pants style incontinence article, it is typically torn apart along at least one of its seams 211 before rolling it up so that it does not have to be removed over the legs. The article according to the present disclosure is in the form of disposal tape 202 in the illustrated embodiment. Disposal tape 202 is used to hold a used (soiled) incontinence article in a rolled-up configuration after it has been torn along the seams 211 as shown in FIG. 3B. Although a variety of disposal tape constructions may be useful, in the illustrated embodiment, the disposal tape 202 includes two adjacent first and second tape tab elements 204, 206 separated by slit 236. Each of the first and second tape tab element 204,206 is adhesively attached to a plastically deformable film 205, which is visible in FIG. 3A. More details about this disposal tape construction can be found in Int. Pat. Appl. Pub. No. WO 2007/032965 (Dahm et al.). In the illustrated embodiment, the tape tab elements 204, 206 each comprise a colored, see-through thermoplastic film and second layer comprising a microporous film. Opaque, microporous region 222 and nonporous region 224 appear as two different colors or two different shades of the same color. The nonporous regions 224 are in the form of alphabetical letters in the illustrated embodiment. However, as described above, the nonporous regions can be in the form of a number, picture, symbol, geometric shape, alphabetical letter, bar code, or any combination thereof. Any of these numbers, pictures, symbols, geometric shapes, alphabetical letters, or combination thereof may be part of a company name, logo, brand name, or trademark picture if desired.

In some embodiments, at least a portion of the article of the present disclosure (e.g., the personal hygiene article) includes an elastomeric material. The term “elastomeric” refers to polymers from which films (0.002 to 0.5 mm thick) can be made that exhibit recovery from stretching or deformation. Exemplary elastomeric polymeric compositions which can be used in the segmented multicomponent polymeric films disclosed herein include thermoplastic elastomers such as ABA block copolymers, polyurethane elastomers, polyolefin elastomers (e.g., metallocene polyolefin elastomers), polyamide elastomers, ethylene vinyl acetate elastomers, and polyester elastomers. An ABA block copolymer elastomer generally is one where the A blocks are polystyrenic, and the B blocks are conjugated dienes (e.g., lower alkylene dienes). The A block is generally formed predominantly of substituted (e.g, alkylated) or unsubstituted styrenic moieties (e.g., polystyrene, poly(alphamethylstyrene), or poly(t-butylstyrene)), having an average molecular weight from about 4,000 to 50,000 grams per mole. The B block(s) is generally formed predominantly of conjugated dienes (e.g., isoprene, 1,3-butadiene, or ethylene-butylene monomers), which may be substituted or unsubstituted, and has an average molecular weight from about 5,000 to 500,000 grams per mole. The A and B blocks may be configured, for example, in linear, radial, or star configurations. An ABA block copolymer may contain multiple A and/or B blocks, which blocks may be made from the same or different monomers. A typical block copolymer is a linear ABA block copolymer, where the A blocks may be the same or different, or a block copolymer having more than three blocks, predominantly terminating with A blocks. Multi-block copolymers may contain, for example, a certain proportion of AB diblock copolymer, which tends to form a more tacky elastomeric film segment. Other elastomers can be blended with block copolymer elastomers provided that the elastomeric properties are not adversely affected. Many types of thermoplastic elastomers are commercially available, including those from BASF under the trade designation “STYROFLEX”, from Shell Chemicals under the trade designation “KRATON”, from Dow Chemical under the trade designation “PELLETHANE” or “ENGAGE”, from DSM under the trade designation “ARNITEL”, from DuPont under the trade designation “HYTREL”, and more. The thermoplastic elastomers including tetrablock styrene/ethylene-propylene/styrene/ethylene-propylene described in U.S. Pat. No. 6,669,887 (Hilston et al.) may also be useful.

For any of the embodiments of the article according to and/or made according to the present disclosure, the tape may be in the form of a roll, from which smaller pieces (for example, tape tabs) may be cut in a size appropriate to the desired application. In this application, the tape may also be a tape tab that has been cut to a desired size, and the method of making a tape can include cutting the tape to a desired size. In some embodiments, including embodiments in which the article is a mechanical fastener, the second surface of the mechanical fastener (i.e., the surface opposite the first surface from which the mechanical fastening elements project) may be coated with an adhesive (e.g., a pressure sensitive adhesive). In such embodiments, when the mechanical fastener is in the form of a roll, a release liner may be applied to the exposed adhesive.

In some of these embodiments, the fastening tab or patch that has been cut from the tape or mechanical fastener roll as described above can be incorporated into personal hygiene article. The tape tab can be attached to a personal hygiene article, for example, by thermal lamination, adhesives (e.g., pressure sensitive adhesives), or other bonding methods (e.g., ultrasonic bonding, compression bonding, or surface bonding).

Another embodiment of the article of the present disclosure is shown in FIG. 4. FIG. 4 is a side cross-section view of a roll of tape 300. First layer 301 is a see-through, thermoplastic film having a first color. That is, the first layer is not colorless and has a color other than white. The first layer has a first major surface 301a and second major surface 301b. The second layer 302 of the roll of tape 300 is adjacent the second major surface 301b of the first layer 301. The second layer 302 comprises a microporous film that has an opaque, microporous region and nonporous regions, which are not shown in the side view of FIG. 4. In some embodiments, including the embodiment illustrated in FIG. 4, the roll of tape 300 including the article of the present disclosure further comprises a third layer 303 adjacent a major surface of the second layer 302 opposite the first layer 301. The third layer 303 has a second color different from the first color. That is, the third layer 303 is also not colorless and has a color other than white. Although not shown in FIG. 4, when the opaque, microporous region and the nonporous region are seen through the first layer 301 they create an appearance on the outer surface of the article of two different colors. While FIG. 4 illustrates the third layer 303, the third layer need not be present in the article of the present disclosure. Roll of tape 300 further includes a release coating 307 on the first major surface 301a of the first layer 301 and an adhesive 309 on the tape on a surface 300s opposite the release coating. The release coating 307 can be provided, for example, by a fluorochemical, silicone, or carbamate. The adhesive 309 can be a pressure sensitive adhesive as described above in any of its embodiments.

In some embodiments, an article of the present disclosure may be joined to a carrier. The article may be joined to a carrier by lamination (e.g., extrusion lamination), adhesives (e.g., pressure sensitive adhesives), or other bonding methods (e.g., ultrasonic bonding, compression bonding, or surface bonding). In some embodiments, the article may be joined to a carrier during the formation of the multilayer film, and stretching to induce or enhance microporosity can be carried out after the multilayer film is joined to a carrier. The resulting article may be a fastening laminate, for example, a fastening tape tab joined to the backsheet of a personal hygiene article useful for joining the front waist region and the rear waist region.

In some embodiments, the carrier for the article of the present disclosure may comprise a variety of suitable materials. For example, the tape backing or carrier may comprise woven webs, non-woven webs (e.g., spunbond webs, spunlaced webs, airlaid webs, meltblown web, and bonded carded webs), textiles, plastic films (e.g., single- or multilayered films, coextruded films, laterally laminated films, or films comprising foam layers), and combinations thereof. In some embodiments, the carrier is a fibrous material (e.g., a woven, nonwoven, or knit material). In some embodiments, the carrier comprises multiple layers of nonwoven materials with, for example, at least one layer of a meltblown nonwoven and at least one layer of a spunbonded nonwoven, or any other suitable combination of nonwoven materials. For example, the carrier may be a spunbond-meltbond-spunbond, spunbond-spunbond, or spunbond-spunbond-spunbond multilayer material. Or, the carrier may be a composite web comprising any combination of nonwoven layers and dense film layers. The carrier may be continuous (i.e., without any through-penetrating holes) or discontinuous (e.g. comprising through-penetrating perforations or pores) and may be colored.

Fibrous materials that provide useful carriers may be made of natural fibers (e.g., wood or cotton fibers), synthetic fibers (e.g., thermoplastic fibers), or a combination of natural and synthetic fibers. Exemplary materials for forming thermoplastic fibers include polyolefins (e.g., polyethylene, polypropylene, polybutylene, ethylene copolymers, propylene copolymers, butylene copolymers, and copolymers and blends of these polymers), polyesters, and polyamides. The fibers may also be multi-component fibers, for example, having a core of one thermoplastic material and a sheath of another thermoplastic material.

Useful carriers may have any suitable basis weight or thickness that is desired for a particular application. For a fibrous tape backing or carrier, the basis weight may range, e.g., from at least about 20, 30, or 40 grams per square meter, up to about 400, 200, or 100 grams per square meter. The carrier may be up to about 5 mm, about 2 mm, or about 1 mm in thickness and/or at least about 0.1, about 0.2, or about 0.5 mm in thickness.

One or more zones of the carrier may comprise one or more elastically extensible materials extending in at least one direction when a force is applied and returning to approximately their original dimension after the force is removed. The term “elastic” refers to any material that exhibits recovery from stretching or deformation. Likewise, “nonelastic” materials, which do not exhibit recovery from stretching or deformation, may be useful for the tape backing or carrier as well.

In some embodiments where the carrier includes a fibrous web, joining thermoplastic components such as the article of the present disclosure to a carrier comprises impinging heated gaseous fluid (e.g., ambient air, dehumidified air, nitrogen, an inert gas, or other gas mixture) onto a first surface of the fibrous web while it is moving; impinging heated fluid onto a surface of the multilayer film while the continuous web is moving, wherein in some embodiments, the surface of the multilayer film is the surface opposite the first surface having mechanical fastening elements; and contacting the first surface of the fibrous web with the surface of the multilayer film so that the first surface of the fibrous web is melt-bonded (e.g., surface-bonded or bonded with a loft-retaining bond) to the multilayer film. Impinging heated gaseous fluid onto the first surface of the fibrous web and impinging heated gaseous fluid on the multilayer film may be carried out sequentially or simultaneously. The term “surface-bonded” when referring to the bonding of fibrous materials means that parts of fiber surfaces of at least portions of fibers are melt-bonded to the surface of the multilayer film in such a manner as to substantially preserve the original (pre-bonded) shape of the surface of the multilayer film, and to substantially preserve at least some portions of the surface of the multilayer film in an exposed condition, in the surface-bonded area. Quantitatively, surface-bonded fibers may be distinguished from embedded fibers in that at least about 65% of the surface area of the surface-bonded fiber is visible above the surface of the multilayer film in the bonded portion of the fiber. Inspection from more than one angle may be necessary to visualize the entirety of the surface area of the fiber. The term “loft-retaining bond” when referring to the bonding of fibrous materials means a bonded fibrous material comprises a loft that is at least 80% of the loft exhibited by the material before, or in the absence of, the bonding process. The loft of a fibrous material as used herein is the ratio of the total volume occupied by the web (including fibers as well as interstitial spaces of the material that are not occupied by fibers) to the volume occupied by the material of the fibers alone. If only a portion of a fibrous web has the surface of the multilayer film bonded thereto, the retained loft can be easily ascertained by comparing the loft of the fibrous web in the bonded area to that of the web in an unbonded area. It may be convenient in some circumstances to compare the loft of the bonded web to that of a sample of the same web before being bonded, for example, if the entirety of fibrous web has the surface of the multilayer film bonded thereto. The hot air should be limited so that it does not form a see-through region in the bonding area unless it is desired. Methods and apparatus for joining a continuous web to a fibrous carrier web using heated gaseous fluid may be found in U.S. Pat. Appl. Pub. Nos. 2011-0151171 (Biegler et al.) and 2011-0147475 (Biegler et al.).

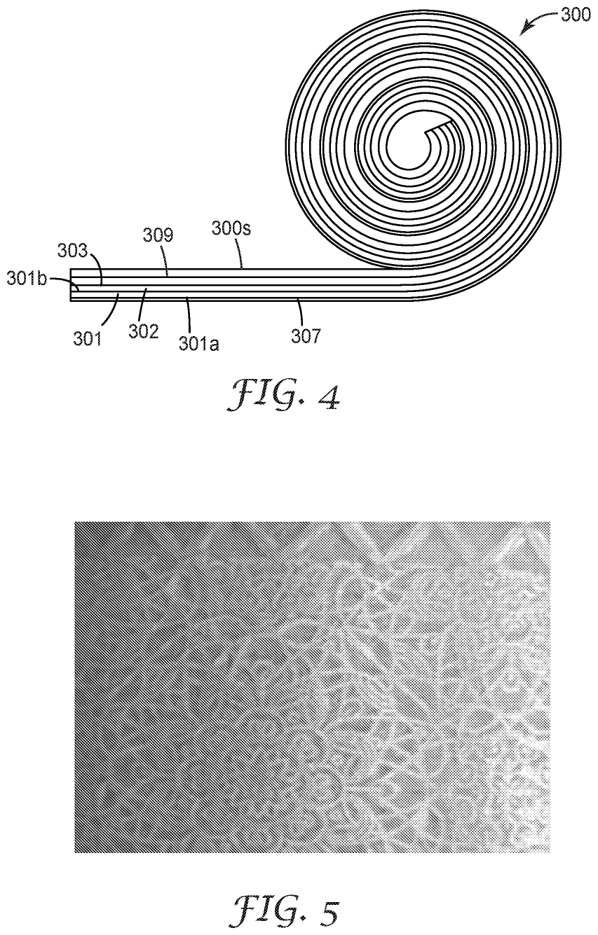

A photograph of an embodiment of an article according to the present disclosure is shown in FIG. 5. In this embodiment, the first surface of the first layer includes upstanding posts, which are male fastening elements. The article has a pattern of nonporous regions within an opaque, microporous region which appears as two different colors. The color of the nonporous regions appears as a combination of the colors of the first layer and the third layer (not shown).

### Some Embodiments of the Disclosure

In a first embodiment, the present disclosure provides an article comprising:

a first layer having first and second major surfaces, wherein the first layer is a see-through thermoplastic film and has a first color other than white; and

a second layer adjacent the second major surface of the first layer, wherein the second layer comprises a microporous thermoplastic film having an opaque, microporous region and a nonporous region,

wherein the first major surface of the first layer is at least a portion of a visible, outer surface of the article, and wherein the opaque, microporous region and the nonporous region seen through the first layer create an appearance on the outer surface of two different colors or two different shades of the same color.

In a second embodiment, the present disclosure provides the article of the first embodiment, wherein the nonporous region is included in a pattern of nonporous regions, which when seen through the first layer create the appearance on the outer surface of a pattern of two different colors or two different shades of the same color.

In a third embodiment, the present disclosure provides the article of the first or second embodiment, wherein the nonporous region is in the form of a number, symbol, picture, geometric shape, bar code, or an alphabetical letter.

In a fourth embodiment, the present disclosure provides the article of any one of the first to third embodiments, further comprising a third layer adjacent a major surface of the second layer opposite the first layer, wherein the third layer has a second color other than white and other than the first color.

In a fifth embodiment, the present disclosure provides the article of the fourth embodiment, wherein in the nonporous region, the first layer, second layer, and third layer together appear to have a color that is a combination of the first color and the second color.

In a sixth embodiment, the present disclosure provides the article of any one of the first to fifth embodiments, wherein the article is a personal hygiene article comprising a chassis with a topsheet, a backsheet, an absorbent component between the topsheet and the backsheet.

In a seventh embodiment, the present disclosure provides the personal hygiene article of the sixth embodiment, wherein the personal hygiene article comprises a fastening tab attached to the first longitudinal edge of the chassis in the rear waist region or the front waist region, wherein the fastening tab comprises the article of the present disclosure. The fastening tab may have any combination of the features of the first to fifth embodiments. The personal hygiene article can also be a pants style personal hygiene article including a chassis with a topsheet, a backsheet, an absorbent component between the topsheet and the backsheet, and the fastening tab attached to at least a portion of the backsheet. The fastening tape in this embodiment may be a disposal tape.

In an eighth embodiment, the present disclosure provides the personal hygiene article of the seventh embodiment, wherein the article forms at least a portion of a tape backing of the fastening tab.

In a ninth embodiment, the present disclosure provides the personal hygiene article of any one of the seventh to eighth embodiments, wherein the article forms at least a portion of a release tape on the fastening tab.

In a tenth embodiment, the present disclosure provides the personal hygiene article of any one of the seventh to ninth embodiments, wherein the article forms at least a portion of a mechanical fastener on the fastening tab.

In an eleventh embodiment, the present disclosure provides a personal hygiene article comprising a chassis with a topsheet, a backsheet, an absorbent component between the topsheet and the backsheet, and a disposal tape attached to the backsheet, wherein the disposal tape comprises the article of any one of the first to tenth embodiments.

In a twelfth embodiment, the present disclosure provides the personal hygiene article of any one of the sixth to eleventh embodiments, wherein the backsheet of the personal hygiene article comprises the first layer and the second layer.

In a thirteenth embodiment, the present disclosure wherein the article is a roll of tape, further comprising a release coating on the first major surface of the first layer and an adhesive on the tape on a surface opposite the release coating.

In a fourteenth embodiment, the present disclosure provides the article of the thirteenth embodiment, wherein the release coating is a silicone, fluorochemical, or carbamate coating.

In a fifteenth embodiment, the present disclosure provides the article of the thirteenth or fourteenth embodiment, wherein the adhesive is a pressure sensitive adhesive.

In a sixteenth embodiment, the present disclosure provides the article of the fifteenth embodiment, wherein the pressure sensitive adhesive comprises an acrylic resin.

In a seventeenth embodiment, the present disclosure provides the article of the fifteenth embodiment, wherein the pressure sensitive adhesive comprises natural or synthetic rubber.

In an eighteenth embodiment, the present disclosure provides the article of any one of the thirteenth to seventeenth embodiments, further comprising a mechanical fastener attached to the adhesive.

In a nineteenth embodiment, the present disclosure provides the article of any one of the first to eighteenth embodiments, wherein the microporous, thermoplastic film comprises a beta-nucleating agent.

In a twentieth embodiment, the present disclosure provides the article of any one of the first to nineteenth embodiments, wherein the microporous, thermoplastic film comprises a diluent.

In a twenty-first embodiment, the present disclosure provides the article of any one of the first to twentieth embodiments, wherein the microporous, thermoplastic film comprises at least one of propylene homopolymer, a copolymer of propylene and other olefins, or a blend of a polypropylene homopolymer and a different polyolefin.

In a twenty-second embodiment, the present disclosure provides a method of making the article of any one of the first to twenty-first embodiments, the method comprising:

providing a multilayer film comprising the first layer and a microporous thermoplastic film; and

collapsing some pores in the microporous thermoplastic film to form the nonporous region.

In a twenty-third embodiment, the present disclosure provides the method of the twenty-second embodiment, further comprising stretching a thermoplastic film comprising at least one of a beta-nucleating agent or a diluent to form the microporous thermoplastic film.

In a twenty-fourth embodiment, the present disclosure provides the method of the twenty-third embodiment, wherein providing the microporous thermoplastic film comprises melt blending a crystallizable polymer and a diluent and cooling to a temperature at which the polymer crystallizes and phase separates from the diluent.

In a twenty-fifth embodiment, the present disclosure provides the method of the any one of the twenty-second to twenty-fourth embodiments, wherein collapsing some pores in the microporous thermoplastic film comprises heating the microporous thermoplastic film to collapse the pores to form the nonporous.

In a twenty-sixth embodiment, the present disclosure provides the method of the twenty-fifth embodiment, wherein heating the microporous thermoplastic film is carried out with a heated, patterned roller.

In a twenty-seventh embodiment, the present disclosure provides the method of the twenty-sixth embodiment, wherein heating the microporous film is carried out with hot air.

In a twenty-eighth embodiment, the present disclosure provides the method of the twenty-sixth embodiment, wherein heating the microporous film is carried out with a laser.

In a twenty-ninth embodiment, the present disclosure provides the method of the twenty-eighth embodiment, wherein the heating with the laser is adjusted to a location of the microporous thermoplastic film within the multilayer film.

In a thirtieth embodiment, the present disclosure provides the method of any one of the twenty-second to twenty-ninth embodiments, further comprising incorporating a portion of the article into a personal hygiene article.

### EXAMPLES

### Preparative Example

A film with upstanding posts was prepared by feeding two streams of polymer through an extrusion die to create a two layer cast film. The streams were extruded at throughput rates to create streams of approximately equal thickness. The layer predominantly on the side with the upstanding posts is referred to as side A and the layer predominantly on the side of the base film is referred to as side B.

Side A comprises of a polypropylene impact copolymer, obtained from Total Petrochemical and Refining USA, under the trade designation “5571 Polypropylene Copolymer” (94 weight %), a pigment masterbatch of pantone 2635u (4 weight %), obtained from Clariant Corp, and a beta nucleating master batch obtained from the Mayzo Corporation under the trade designation “MPM 2000” (2 weight %) extruded through a 40 mm twin screw extruder. Seven barrel zones in the extruder were set at 160° C., 170° C., 180° C., 190° C., 200° C., 220° C., and 220° C., respectively. The molten resin was then fed through a two layer feed block set at 220° C. and sheet die set at 220° C.

Side B comprises of a polypropylene impact copolymer, obtained from Total Petrochemical and Refining USA, under the trade designation “5571 Polypropylene Copolymer” (98 weight %), and a beta nucleating master batch obtained from the Mayzo Corporation under the trade designation “MPM 2000” (2 weight %) extruded through a 1½ inch single screw extruder. Five barrel zones in the extruder were set at 375° F. (191° C.), 390° F. (199° C.), 400° F. (204° C.), 415° F. (213° C.), and 425° F. (218° C.), respectively. The molten resin was then fed through a two layer feed block combining with side A in the feedblock and exiting the sheet die as a two layer film to a rotating cylindrical mold. The temperature of cylindrical mold was set at 93° C.

The post density was 3500 posts per square inch arranged in a staggered array and the post shape was conical. The cross-sectional shape of the post at the base was circular with a diameter of 175 micrometers and the post height was 350 micrometers. The line speed was set such that the film basis weight was 110 grams per square meter. The web was fed into a cap forming apparatus after slitting it to the width to fit the apparatus. The posts were capped with oval shaped caps using the procedure described in U.S. Pat. No. 5,845,375 (Miller et al.). The film was then stretched in the machine direction by passing the web through two rolls in which one roll was rotating faster than the other one. The bottom roll was a chrome roll, and the top roll was a rubber roll. For stretching, the temperature of the bottom chrome roll was set at 165° F. (74° C.) and the top rubber roll was set at 165° F. (74° C.). The draw ratio was 2.5:1 in the machine direction.

The film was subsequently run through a two roller in-running nip. The top roller was a rubber coated steel roller with a pattern in the rubber surface. The roller was internally heated with water and set at 200° F. (93° C.). The bottom roller was a chrome plated steel roller with a smoother surface. The roller was internally heated with heat transfer oil and the oil temperature was adjusted to give a surface temperature of 315° F. (157° C.). With the post side of the film facing the rubber coated roller, the film was embossed with a nip pressure of 12 kN creating a two-toned film where the microporous portions of the film were condensed in the areas of the film that was exposed to the nip force between the patterned section on the rubber roller and the chrome roller.

### Example

A two-colored film was created using the film from the Preparative Example. The film was run through a two roller in-running nip. The top roller was a rubber coated steel roller with a pattern in the rubber surface. The roller was internally heated with water and set at 200° F. (93° C.). The bottom roller was a chrome plated steel roller with a smoother surface. The roller was internally heated with heat transfer oil and the oil temperature was adjusted to give a surface temperature of 325° F. (163° C.). With the post side of the film facing the rubber coated roller, the film was embossed with a nip pressure of 15 kN creating a two-toned film where the microporous portions of the film were condensed in the areas of the film that was exposed to the nip force between the patterned section on the rubber roller and the chrome roller. The condensed area had a translucent purple tint. The backing side of a two-inch-wide by ten-inch-long strip was laminated to a strip of commercially available 3M Vinyl Tape 471 Yellow. When looking at the film from the post side, the condensed areas show as a combination of the translucent purple film and the yellow film from the laminated tape.

This disclosure may take on various modifications and alterations without departing from its spirit and scope. Accordingly, this disclosure is not limited to the above-described embodiments but is to be controlled by the limitations set forth in the following claims and any equivalents thereof. This disclosure may be suitably practiced in the absence of any element not specifically disclosed herein.

1. An article comprising:
a first layer having first and second major surfaces, wherein the first layer is a see-through thermoplastic film and has a first color other than white; and
a second layer adjacent the second major surface of the first layer, wherein the second layer comprises a microporous thermoplastic film having an opaque, microporous region and a nonporous region,
wherein the first major surface of the first layer is at least a portion of a visible, outer surface of the article, and wherein the opaque, microporous region and the nonporous region seen through the first layer create an appearance on the outer surface of two different colors or two different shades of the same color.

2. The article of claim 1, wherein the nonporous region is included in a pattern of nonporous regions, which when seen through the first layer create the appearance on the outer surface of a pattern of two different colors or two different shades of the same color.

3. The article of claim 1, wherein the nonporous region is in the form of a number, symbol, picture, geometric shape, bar code, an alphabetical letter, or a combination thereof.

4. The article of claim 1, further comprising a third layer adjacent a major surface of the second layer opposite the first layer, wherein the third layer has a second color other than white and other than the first color.

5. The article of claim 4, wherein in the nonporous region, the first layer, second layer, and third layer together appear to have a color that is a combination of the first color and the second color.

6. The article of claim 1, wherein the microporous thermoplastic film comprises at least one of a beta-nucleating agent or a diluent.

7. The article of claim 1, wherein the first layer comprises upstanding posts on the first major surface.

8. The article of claim 1, wherein the article is a roll of tape, further comprising a release coating on the first major surface of the first layer and an adhesive on the tape on a surface opposite the release coating.

9. The article of claim 1, wherein the article is a personal hygiene article comprising a chassis with a topsheet, a backsheet, and an absorbent component between the topsheet and the backsheet.

10. The article of claim 9, wherein the backsheet of the personal hygiene article comprises the first layer and the second layer.

11. A method of making the article of claim 1, the method comprising:
providing a multilayer film comprising the first layer and a microporous thermoplastic film; and
collapsing some pores in the microporous film to form the nonporous region.

12. The method of claim 11, further comprising stretching a thermoplastic film comprising at least one of a beta-nucleating agent or a diluent to form the microporous thermoplastic film.

13. The method of claim 12, further comprising coextruding the first layer and the thermoplastic film comprising at least one of the beta-nucleating agent or the diluent, wherein the stretching comprises stretching the first layer and the thermoplastic film comprising at least one of the beta-nucleating agent or the diluent simultaneously.

14. The method of claim 11, wherein collapsing some pores in the microporous film comprises heating the microporous film to collapse the pores to form the at least one see-through region of lower porosity.

15. The method of claim 14, wherein heating the microporous film is carried out with a heated, patterned roller, with hot air, or with a laser.

16. The method of claim 11, further comprising incorporating a portion of the article into a personal hygiene article.

17. The article of claim 9, wherein the personal hygiene article further comprises a fastening tab attached to a first longitudinal edge of the chassis in a rear waist region or a front waist region, wherein the fastening tab comprises the first layer and the second layer.

18. The article of claim 17, wherein the first layer and the second layer form at least a portion of a tape backing of the fastening tab.

19. The article of claim 17, wherein the first layer and the second layer form at least a portion of a release tape on the fastening tab.

20. The article of claim 9, wherein the personal hygiene article further comprises a disposal tape attached to the backsheet, wherein the first layer and the second layer form at least a portion of the disposal tape.
